(19) United States
(12) Patent Application Publication
(10) Pub. No.: US 2004/0148105 A1
(43) Pub. Date: Jul. 29, 2004

(54) Computational method for predicting protein interaction sites

(75) Inventors: Mary Jo Ondrechen, Hopkinton, MA (US); James G. Clifton, Attleboro, MA (US); Dagmar Ringe, Belmont, MA (US); Ronald J. Williams, Wellesley, MA (US); Robert P. Futrelle, Newton Center, MA (US); Wenxu Tong, Nashua, NH (US); David E. Budil, Stoughton, MA (US); Jaeju Ko, Indiana, PA (US); Leonel F. Murga, Quincy, MA (US); Ihsan A. Shehadi, Watertown, MA (US); Huyuan Yang, Canton, MA (US)

Correspondence Address: WEINGARTEN, SCHURGIN, GAGNEBIN & LEBOVICI LLP, TEN POST OFFICE SQUARE, BOSTON, MA, 02109, US

(73) Assignee: Northeastern University

(21) Appl. No.: 10/678,756

(22) Filed: Oct. 2, 2003

Related U.S. Application Data
(60) Provisional application No. 60/415,742, filed on Oct. 3, 2002.

(51) Int. Cl. G06F 19/00; G01N 33/48; G01N 33/50

(52) U.S. Cl. 702/19

(57) ABSTRACT

A computational method that predicts chemical and electrostatic properties of residues in proteins and utilizes information contained in those predictions to identify various interaction sites is disclosed. The various interaction sites may include, for example, cofactor binding sites, ligand binding sites, catalytic (active) sites or recognition sites. The method of the invention identifies the ionizable residues in the protein with anomalous predicted titration behavior and searches for the clustering of those residues into putative interaction sites. Practicing the method of the invention requires only the structure of the subject protein (which may be deduced, a priori, from the amino acid sequence) and, thus, may be applied to proteins that bear no similarity in structure or sequence to any previously characterized protein.

### CROSS REFERENCE TO RELATED APPLICATIONS

This application claims priority from U.S. Provisional Application No. 60/415,742, filed on Oct. 3, 2002, entitled THEMATICS: A SIMPLE COMPUTATIONAL METHOD FOR THE IDENTIFICATION AND CHARACTERIZATION OF ENZYME ACTIVE SITES, the whole of which is hereby incorporated by reference herein.

### STATEMENT REGARDING FEDERALLY SPONSORED RESEARCH OR DEVELOPMENT

Part of the work leading to this invention was carried out with United States Government support provided under grants from the National Science Foundation, Grant Nos. MCB-0135303, MCB-0074574 and CHE-9974642. Therefore, the U.S. Government has certain rights in this invention.

### BACKGROUND OF THE INVENTION

Knowledge of protein sequences and structures has burgeoned very recently as a result of genome sequencing (Birney et al. 2001) and structural genomics efforts. In order to translate such information into tangible benefits for humankind, the next step is to develop methods that enable one to predict and to establish function from structure. This need becomes particularly acute as novel protein folds are discovered for which there are no proteins of similar structure with known function.

For some classes of proteins, information about function can be inferred from the evolutionary history derived from sequence relationships (Lichtarge et al. 1966; Sjolander et al. 1998; Yao et al. 2003), or by other homology and non-homology sequence methods (Marcotte et al. Science 1999; Marcotte et al. Nature 1999; Marcotte 2000; Ramani et al. 2003). Combined analysis of sequence and structure data gives more revealing clues about function (Carter et al. 2001). Current methods to locate active sites rely either on analogies to related proteins of known function (Babbitt et al. 1998; Fetrow et al. 2001; Fetrow et al. 1999; Fetrow et al. 1998; Hegyi et al. 1999; Skolnick et al. 2000; Teichmann et al. 2001; Wallace et al. 1997), on searches for clefts in the structure (Laskowski et al. 1996), or on computational searches for binding sites by docking (Park et al. 2000) of selected sets of small molecules (Chen 2001; Chen et al. 2001; Dennis et al. 2002). Energetics (Elcock 2001) and flexibility (Ma et al. 2001) can also predict functionally important sites. However, there is as yet no reliable method to identify active sites of enzymes or other interaction sites of proteins in the absence of biochemical data, even when the structure is known.

### BRIEF SUMMARY OF THE INVENTION

The method of the invention, Theoretical Microscopic Titration Curves (THEMATICS), is a computational method that predicts chemical and electrostatic properties of residues in proteins and utilizes information contained in those predictions to identify various interaction sites. The various interaction sites may include, for example, cofactor binding sites, ligand binding sites, catalytic (active) sites or recognition sites. The method of the invention identifies the ionizable residues in the protein with anomalous predicted titration behavior and searches for the clustering of those residues into putative interaction sites. Practicing the method of the invention requires only the structure of the subject protein (which may be deduced, a priori, from the amino acid sequence) and, thus, may be applied to proteins that bear no similarity in structure or sequence to any previously characterized protein. To predict functional information from the primary sequence, one starts with the primary amino acid sequence as input and predicts the three-dimensional structure theoretically, using a modeling method including but not limited to comparative modeling, homology modeling, and threading. Then, one applies the method of the invention to the theoretical three-dimensional structure as described.

In practicing the method of the invention, the mean net charge C, averaged over the ensemble of protein molecules, is determined as a function of pH for each of the ionizable residues in the protein structure. The resulting C(pH) curves represent the theoretical microscopic titration curves for each ionizable species in the protein structure. The majority of these C(pH) curves in a given protein structure have the typical sigmoidal shape, as predicted by the Henderson-Hasselbalch equation. However, a residue in a given protein may show an unusual shape in the predicted C(pH) function, indicating that partial protonation persists over a wide pH range. The identification of two or more residues with unusually shaped or non-sigmoidal predicted C(pH) curves is utilized to identify a positive cluster, or a predicted site of interaction in a protein.

Thus, in a preferred embodiment, the invention is directed to obtaining a three-dimensional structure of the protein, calculating an electrical potential function for the protein structure, calculating a titration curve for each ionizable residue in the protein, evaluating the shape of the titration curve for each residue, identifying any residue with a perturbed titration curve in comparison with other residues of the same kind, and identifying any residues with perturbed titration curves that are in a cluster. The existence of a cluster indicates that the residues in the cluster are at an interaction site in the protein. The method may further include step of identifying any residue with a perturbed titration curve that does not fall into any cluster.

### BRIEF DESCRIPTION OF THE FIGURES

Other features and advantages of the invention will be apparent from the following description of the preferred embodiments thereof and from the claims, taken in conjunction with the accompanying drawings, in which:

FIG. 1 shows titration curves, which is the predicted mean net charge as a function of pH, for tyrosine residues Y225-Y284 of the A-chain of alanine racemase (Y225 (▪), Y239 (◯), Y265(▴), Y269(∇), Y284(♦));

FIG. 2 shows titration curves for histidine residues in the A-chain of triosephosphate isomerase (His-26(+), His-95(×), His-100(*), His-115(□), His-185(▪), His-195(◯), His-224(●), and His-248(Δ));

FIG. 3 shows a ribbon diagram of triosephosphate isomerase protein structure, looking down the α/β barrel at the active site (backbone is shown in light gray; active-site residues with perturbed titration curves are shown in black and labeled);

FIG. 4 shows a ribbon diagram of aldose reductase protein structure, looking down the α/β barrel at the active site (NADPH cofactor is shown in medium gray) (backbone is shown in light gray; active-site residues with perturbed titration curves are shown in black and labeled);

FIG. 5 shows titration curves for selected tyrosine residues for aldose reductase (Tyr-39(+), Tyr-48(×), Tyr-177(*), Tyr-291(□), and Tyr-309(▪));

FIG. 6 shows titration curves for selected lysine residues for phosphomannose isomerase (Lys-100(+), Lys-117(×), Lys-128(*), Lys-136(□), and Lys-153(▪));

FIG. 7 shows a ribbon diagram phosphomannose isomerase, looking down at the presumptive active site (zinc ion is shown as a medium gray ball) (backbone is shown in light gray; active-site residues with perturbed titration curves are shown in black and labeled);

FIG. 8 shows titration curves for lysine residues from 144 through 355 of the A-chain of the template structure for aspartate aminotransferase (K144 (+), K215 (×), K248 (*), K258 (□), K288 (▪), K344 (◯), K355 (●)); and

FIG. 9 shows a ribbon diagram of papain, showing the side chains of the THEMATICS positive residues C25 and H159 in the active site in red, the second cluster K17, K174 and Y186 in yellow, and the isolated residues E52 and R96 each in blue.

### DETAILED DESCRIPTION OF THE INVENTION

The method of the invention is directed to predicting protein function from sequence and structure information, even in the absence of biochemical data. Theoretical Microscopic Titration Curves (THEMATICS) is a computational method that predicts the chemical and electrostatic properties of residues in enzymes or other proteins and uses that information to determine the functional activity at the atomic and molecular level of a given protein. THEMATICS makes use of theoretical microscopic titration curves calculated from the electrical potential function for all of the ionizable residues in the protein structure. THEMATICS searches for residues with anomalous shapes in their theoretical microscopic titration curves and then seeks clusters of such residues in coordinate space. Most chemical reactivity in proteins depends on residues that can function as either Brønsted or Lewis acids and bases, and, therefore, such reactive residues are expected to show anomalous titration behavior. In this manner, THEMATICS locates regions within the protein structure where chemical reactivity (or interaction of any kind) is likely to occur. This method has proved to be a very reliable predictor of the location of interaction site(s), given the structure of an enzyme, whether that structure is known or determined theoretically from the amino acid sequence of the protein. The various sites of activity may include, but are not limited to, catalytic sites, recognition sites, metal binding sites, cofactor binding sites and ligand binding sites.

One of the advantages of the present method is its simplicity. It is computationally fast, is not database dependent and is amenable to automation for high-volume computational screening. Thus, the method of the invention will be a particularly useful tool for the biotechnology and drug industries, e.g., for screening proteins for target sites for the potential binding of drugs and other effector molecules.

This method requires only the structure of the subject protein and thus complements the database-dependent approaches. The protein does not have to bear any resemblance in sequence or structure to any previously characterized protein for THEMATICS to be applicable. Based on the evidence presented, the method of the invention is also applicable to proteins for which an experimentally determined structure is unavailable, i.e., where a theoretical structure is determined from the amino acid sequence of the protein.

Therefore, in practice of the method of the invention, a three-dimensional structure of the protein is first obtained. This structure can be determined experimentally (generally by x-ray diffraction or NMR) or downloaded from a database. The structure can also be a theoretical model structure; in this case, the initial input to the method is the primary sequence of the protein. The three-dimensional structure of the protein is then predicted theoretically, using a modeling method including but not limited to comparative modeling, homology modeling, and threading. Then, one applies the method of the invention to the theoretical three-dimensional structure as described.

Once the model of the protein has been obtained, the electrical potential function is calculated for the protein structure using standard methods in the art. There are a number of programs available for carrying out such calculations. These programs generally utilize a Poisson-Boltzmann (Yang et al. 1993; Bashford et al. 1991; Warwicker et al. 1982; Antosiewicz et al. 1996) procedure and solve the equations with a finite difference method or a finite element method. Exemplary programs that calculate the electrical potential function for proteins include UHBD (Madura et al. 1995), DelPhi (http://www.accelrys.com/insight/DelPhi page.html), APBS (Adaptive Poisson Boltzmann Solver, which uses a finite element method) (Baker et al. 2001) and WHATIF: a molecular modeling and drug design program (Vriend 1990).

Next, the titration curves for all of the ionizable residues in the protein are calculated, using standard procedures in the art. Exemplary methods include, but are not limited to, the full Boltzmann sum method (Bashford et al. 1991), a hybrid Tanford-Roxby/Boltzmann method (Yang et al. 1993; Karshikoff 1995), and a Monte Carlo sampling method (Beroza et al. 1991). Programs that are available to perform these calculations include HYBRID (Antosiewicz et al. 1996; Gilson 1993) and WHATIF (Vriend 1990). One of these methods is used to calculate the mean net charge as a function of pH for each ionizable group (all lysine, arginine, aspartic acid, glutamic acid, histidine, tyrosine and cysteine residues, and the N-terminus and C-terminus) of each protein.

Finally, the shapes of the predicted titration curves are compared and evaluated for each residue of the same kind (e.g., among all lysine residues, identifying the specific one(s) with anomalous shaped curves in comparison with all the rest). Most of the curves have a sigmoidal shape, as predicted by the Henderson-Hasselbalch equation, and the anomalous residues, whose predicted titration curves have a non-sigmoidal shape, are identified. These anomalous residues have elongated titration curves, such that partial protonation is predicted to extend over a larger pH range than for a more typical residue. The wide pH range is one that exceeds the range of stability of most protein structures. The titration functions are used as a diagnostic tool and not an implication that these extremes of pH could actually be achieved. We have developed three ways of identifying the residues with anomalous predicted titration behavior: (a) simple visual inspection of the curves by human observation; (b) statistical analysis of the curve features to identify the curves that are outliers; and (c) automated classification. Residues with anomalous predicted titration behavior are called THEMATICS positive residues.

The anomalous residues located in physical proximity in the protein structure are then identified. A group of anomalous residues in physical proximity is called a THEMATICS positive cluster. Anomalous residues are in physical proximity when two or more are nearest neighbors, or are surrounding the same cleft as another anomalous residue, or have reactive atoms within a certain cut-off distance of each other. Such residues, by definition, belong to the same THEMATICS positive cluster. Distances between residues that are considered to be in physical proximity may be, e.g., less than 15 Å, preferably less than 10 Å, more preferably less than 7 Å, and most preferably approximately 6 Å or less.

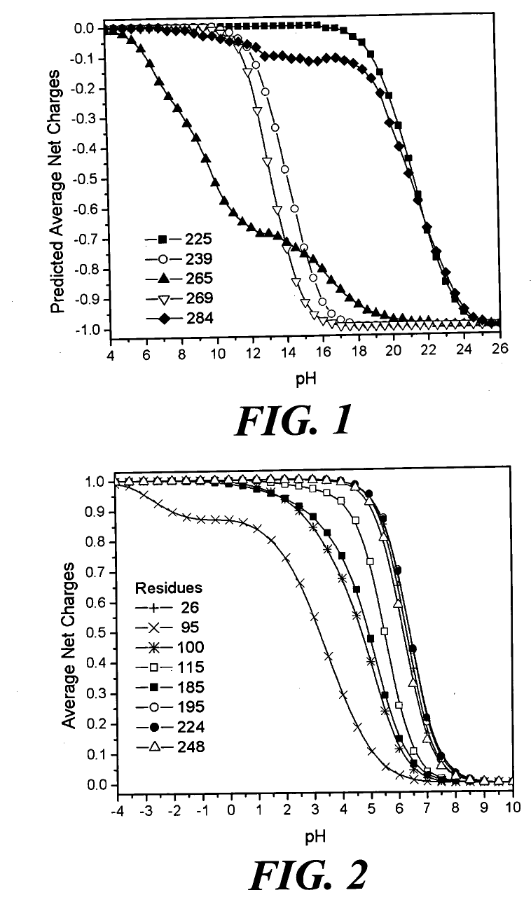

Identification of positive clusters by visual inspection of the curves often works very well, as in the proof-of-principal example shown in FIG. 1. This figure, which presents theoretical titration curves calculated by THEMATICS, shows the predicted average net charge as a function of pH for tyrosine residues at amino acid positions in the range 225-284 in the A chain of alanine racemase, a bacterial enzyme that is a target for antibiotics and that has a known active site. As can be seen, Y239 and Y269 are typical tyrosine residues with predicted titration curves that fit the Henderson-Hasselbach equation; Y225 has an upshifted pKₐ, but it has a nearly-sigmoidal shape; therefore, none of these three is deemed positive and, indeed, none of these is an active site residue in the known structure. The predicted curve shapes for Y265 and Y284, however, are highly a typical and non-sigmoidal; thus, these constitute positive (i.e., anomalous) results. The flattened regions where partial protonation persists over a wide pH range are indicative of a residue that is part of an interaction site. Comparison with a known structure of alanine racemase that contains a substrate-mimic inhibitor reveals that these two residues are, indeed, in the active site; Y265 is a catalytic base and Y284 is involved in substrate recognition (Shaw et al. 1997; Stamper et al. 1998). Y265 and Y284 are part of the known active site cluster [R219, C311, K39, Y43, Y265, Y284, Y354, C358, Y164] that the THEMATICS method correctly predicts for alanine racemase.

A more accurate way of identifying residues in a positive cluster is by a statistical analysis evaluation of the theoretical titration curves. A typical ionizable residue in a protein obeys the Henderson-Hasselbalch equation:

pH=pKₐ+log{[A⁻]/[HA]} (1)

Thus, as pH is increased, the predicted average charge falls sharply in a pH range close to the pKₐ. (The pKₐ of a residue is defined as the pH at which half of the protein molecules in an ensemble have that residue in protonated form HA and half in deprotonated form A⁻.) The ionizable species goes from fully protonated to fully deprotonated in a narrow pH range.

To analyze the calculated titration curves, the Henderson-Hasselbalch equation, equation (1), is rewritten to express the mean net charge C (for a given residue in an ensemble of protein molecules) as a function of pH, as:

C(pH)=10^{pKa}/(10^{pH}+10^{pKa}) (2)

for the residues that form a cation upon protonation (Arg, His, Lys and the N-terminal amino group). Equation (2) is written as:

C(pH)=−10^{pH}/(10^{pH}+10^{pKa}) (3)

for the residues that form an anion upon deprotonation (Asp, Cys, Glu, Tyr and the C-terminal carboxylate group). Equations (2) and (3) both have the familiar sigmoid shape that is typical of a weak acid or base that obeys the Henderson-Hasselbalch equation.

One of the metrics that is used to characterize the titration curves is the number of pH units over which the residue is partially protonated in the range 0.01≦C≦0.99. This number of pH units, P, may be defined as:

P=pH_{c=0.01}−pH_{c=0.99} (4)

P has the nominal value of 3.99, P_{HH}=2*log₁₀(99)=3.99, for a residue that obeys equation (2) (an ordinary Henderson-Hasselbalch residue). P is evaluated for all of the ionizable residues in each protein. Statistical analysis of the P values for all of the ionizable residues then identifies the residues that have a statistically significant deviation from the average P.

We can also fit the predicted titration curves to sigmoid functions of the form:

C(pH)=10^{s}/(10^{r*pH}+10^{s}) (5)

or:

C(pH)=−10^{r*pH}/(10^{r*pH}+10^{s}) (6)

where r and s have the nominal values of 1.0 and the pKₐ, respectively, for a Henderson-Hasselbalch residue. We then obtain the best-fit values for r and s. For instance, one can use a Levenberg-Marquardt procedure in which the quality of the fit is measured by chi-squared, χ². As one might expect, for the perturbed residues, r tends to deviate from 1.0, s tends to deviate from the pKₐ, and χ² tends to be large (indicative of a poor-quality fit). The values P, r, s and χ² for each ionizable residue can be used as measures of the degree of perturbation, or deviation, from Henderson-Hasselbalch behavior. Again, statistical analysis of r, s and χ² for all of the ionizable residues indicates which residues deviate from normal behavior and, therefore, points to the anomalous residues. A variation of the above procedure uses a one-parameter sigmoidal function, where r is the one parameter and x=pH−pKₐ is the dependent variable.

Another valuable set of metrics that we have developed for the characterization of the titration curves involves a first derivative function f, defined as:

f=−dC/dx, (7)

where x is the deviation from the pKₐ, x=pH−pKₐ. (Note that the first derivative itself is usually negative, since charge almost always decreases with increasing pH, so that f is positive.) The n^{th} moment of a function f may be defined as:

<xⁿ>=∫xⁿf dx (8)

We have analyzed the n^{th} moments of the first derivative function f(x). We found that the second and higher moments, particularly the fourth moment, are well correlated with perturbed titration behavior and thus can be used to identify residues that are likely to be in the active site.

As no one single statistical criterion, however, can identify the positive residues in all proteins, the best method of analyzing the theoretical titration curves is by automated classification. The automated classifier takes as input the results of the statistical analyses described above, or, alternatively, the predicted titration curves themselves, and identifies the anomalous residues using an optimal combination of weighted factors. Thus, the automated classifiers are more powerful and more sophistocated than simple statistical analysis. Two approaches have been developed at the present time for automatic identification of interaction sites in enzymes, given as input the spatial structure of the enzyme. Both involve using trainable classifiers, including but not limited to, neural networks and support vector machines.

In the first approach, the trained classifier is used to replace the human visual inspection step as a means of identifying perturbed curves corresponding to residues likely to be in the active site. Various attributes of the curves are used as input to the classifier, including those described above for the statistical analysis process. The labeled training set for the classifier is derived from proteins for which literature exists identifying sites of activity.

The resulting classifier is then used on proteins for which this data is not yet known, identifying likely candidates for residues belonging to active sites. Finally, this stage is combined with a second stage based on determining the physical proximity of multiple active-site candidates, as described above. The result is then an automatically determined THEMATICS positive cluster, as described earlier.

In the second approach, an alternative one-step classifier is trained that takes as input both the data for all the titration curves on the enzyme and the relative spatial locations of all the corresponding residues. Training and classification in this case requires devising attributes that combine measures of curve perturbation of a given residue with measures of the perturbation of all nearby residues, using the spatial distances to the nearby residues. In the simplest case, one can devise a single score to be assigned to each residue that combines the perturbation score of that residue (as in the first approach) with the perturbation scores of nearby residues weighted according to a function that drops off with distance.

An important potential variant of either approach is to allow varying amounts of human involvement, such as allowing a user to interactively set various thresholds or other classification parameters in order to fine-tune the results produced by the otherwise automated program. This will be of particular importance for more knowledgeable users, for whom these techniques can be used to provide automated assistance in their quest for deeper understanding of specific proteins, e.g., enzymes, they wish to study.

For a given protein molecule, most of the residues giving positive results form a cluster in physical space around the site where enzymatic catalysis occurs. For each of the three featured protein molecules with known active sites presented in Example I-III, the THEMATICS method correctly gives positive results for at least two of the active site residues, and most of the positive results are for what turn out to be active site residues. Less often, positive results are obtained for a residue just outside the active site; these residues are termed second shell residues. These second shell residues, which are in physical proximity to active site residues but are not immediate neighbors of the reacting substrate, might well play important functional roles in catalysis or recognition. Occasionally, an apparent false positive, a residue with a perturbed theoretical titration function that is not located near a known active site, is found. However, these false positive residues tend to be isolated in space and are thus distinguished readily from residues in positive clusters. The majority of positive results do cluster together and do occur in the active site, such that its location can be identified with confidence. These trends hold true for a number of types of enzymes, including six of the seven additional examples of enzymes shown in Table 1.

TABLE 1
Positive Results for Some Additional Examples.

| PDB ID | Name | Chemistry |
|---|---|---|
| 1AMQ | Aspartate | transamination |
| | aminotransferase [44] | |

[H189, Y225, K258, R266, C191, C192, Y256], [Y295], [H301]

| | | |
|---|---|---|
| 1CSE | Subtilisin Carlsberg | peptide hydrolysis (serine protease) |

[D32, H64]

| | | |
|---|---|---|
| 1EA5 | Acetylcholine esterase | ester hydrolysis |

[Y130, E199, E327, H440, D392], [Y148], [H398], [H425]

| | | |
|---|---|---|
| 1HKA | 6-Hydroxymethyl-7,8- | kinase |
| | dihydropterin | |
| | pyrophosphate kinase [44] | |

[D97, H115]

| | | |
|---|---|---|
| 1OPY | δ5-3-Ketosteroid isomerase | isomerase |

[Y16, Y32, Y57], [C81]

| | | |
|---|---|---|
| 1PIP | Papain | peptide hydrolysis (Cys protease) |

[C25, H159], [K17, K174, Y186], [R59], [R96]

| | | |
|---|---|---|
| 1PSO | Pepsin | peptide hydrolysis (acid protease) |

[D32, D215, D303], [D11]

| | | |
|---|---|---|
| 1WBA | Winged bean albumin | storage - no known chemistry |

No positive results
Positive results that form a cluster in coordinate space are shown in brackets.
Active site residues are shown in boldface.
Second shell residues are shown in italics.

For triosephosphate isomerase (TIM) (Example I), the method of the invention yields four positive results. Two of the four (His95 and Glu165) are active site residues known to be involved in catalysis. The third positive result, that for Tyr164, is located right next to the active site, in what might be thought of as the second shell surrounding the reacting substrate. A fourth residue, Lys112, is a false positive. Seven positive results were found for aldose reductase (AR) (Example II): Tyr48 is an active site residue and is known to be involved in catalysis; Cys298, Lys77 and Tyr209 are known active site residues; and Glu185 and Lys21 are located just outside the active site, again in the second shell; Tyr107 we consider to be a false positive. For phosphomannose isomerase (PMI) (Example III), four positive results were found: three (Lys100, Lys136 and Tyr287) are located in the presumed active site; One (His135) is in the second shell of the presumed active site. The total number of ionizable residues equals 77 per chain for TIM, 103 for AR and 134 for PMI. For these proteins, therefore, about 3-7% of the ionizable residues yield a positive result under the present definition.

The calculated pKₐ value for His95 of TIM is consistent with ¹⁵N NMR data (Lodi et al. 1991). The calculated pKₐ of 8.4 for Tyr48 of AR is identical to the value reported in Bohren et al. reference (Bohren et al. 1994). All of the calculations reported here have been performed in the absence of cofactors and substrate mimics, in order to simulate conditions where a structure is built from genomic data and knowledge of the location or identity of such species in the protein structure may not be available. The presence of the NADPH cofactor in AR will cause some shifting of the pKₐs of adjacent residues. Furthermore, the presence of a substrate or substrate mimic can cause substantial shift in the calculated pKₐ of an adjacent residue; this type of behavior was reported recently for alanine racemase (Ondrechen et al. 2001).

A few positive results were found for residues that are presumed to be just outside the active site, or in the second shell. These residues are Tyr164 in TIM, Glu185 and Lys21 in AR and His135 in PMI. It is undetermined whether these residues have perturbed titration functions because they actually participate in catalysis and/or recognition, or because they are simply physically very close to the area where catalysis occurs. In the latter case, they would presumably be subject to the same pH-dependent potentials as the species that are engaged in catalysis. Three out of four of these residues are conserved (Gish et al. 1993). Tyr164 in TIM and His135 in PMI are conserved across a range of species. A ClustalW alignment (http://www.ebi.ac.uk/clustalw) (Thompson et al. 1997) was run for TIM sequences from chicken, human, mouse, Drosophila melanogaster, Zea mays (maize) and Emericella nidulans. His95, Tyr164 and Glu165 are conserved in TIM from all of these species. A ClustalW alignment (see supra) was run for PMI sequences from Candida albicans, human, Saccharomyces cerevisiae, Emericella nidulans, and Salmonella typhimurium. Lys100, His135, Lys136 and Tyr287 are conserved across all of these species. Glu185 in human aldose reductase is conserved over a number of human enzymes with some sequence identity. A ClustalW alignment (see supra) alignment was run for sequences of the human enzymes aldose reductase, bile acid binding protein, chlordecone reductase, 3-oxo-5-beta-steroid reductase and aldehyde reductase. Tyr48, Glu185, Lys77 and Tyr107 are conserved across these enzymes. Cys298 and Lys21 are not conserved. Tyr209 is conserved across all of the proteins except chlordecone reductase, where it is replaced by a histidine. This observed conservation lends support to the notion that these residues do play functional roles, e.g., in catalysis, recognition, catalytic efficiency or in the stabilization of the protein fold.

The question why there are sometimes apparent false positive results is an intriguing one. One possible explanation is that proteins simply have strong electric fields and that occasionally these pH-dependent fields, arising either from long-range forces or from a strongly-coupled neighbor, will just happen to cause perturbations in the titration functions that resemble those of catalytic residues. Another possible explanation is that these residues actually do have some chemical function that has not yet been established. A third possibility is that these residues are a part of a vestigial (or incipient) active site that had (or will have) a function for some past (or future) species. A fourth possibility is that they are artifacts related to the quality of the input atomic coordinates, namely, the interpretation of the electron density maps in a region of some disorder may position residues inaccurately.

THEMATICS calculations on an enzyme of unknown function usually yield one cluster and this cluster is presumed to be the active site. Thus one can identify the active site even if that protein bears no resemblance to any previously characterized protein. However, for a protein of unknown function, to obtain further information about the function, to characterize the sites in that protein, to determine what type of chemistry might be catalyzed by that protein, and to establish where recognition takes place in the protein structure, one needs to compare the results with proteins of known function that have been characterized. For instance, analysis of the results for the protein Kex2 (described in Example IX, below) reveals a cluster that contains the well-known catalytic triad of a hydrolase. One can then hypothesize that the other positive residues that are not a part of that catalytic triad are involved in recognition.

The following examples are presented to illustrate the advantages of the present invention and to assist one of ordinary skill in making and using the same. These examples are not intended in any way otherwise to limit the scope of the disclosure. The contents of all references and pending and published patent applications cited throughout this application are hereby incorporated by reference.

Identification of the Sites of Interaction, using as Examples, Enzymes with Known Active Sites. (EXAMPLES I-III)

### EXAMPLE I

### Triosephosphate Isomerase (TIM)

TIM (Zhang et al. 1994; Coulson et al. 1970; Waley et al. 1970; Hartman 1970; Komives et al. 1991; Lodi et al. 1991) catalyzes the isomerization of D-glyceraldehyde 3-phosphate (GAP) to dihydroxyacetone phosphate (DHAP), a key reaction in the glycolytic pathway, and has the α/β barrel (“TIM barrel”) fold. Calculations on TIM from Gallus gallus (chicken) were performed using the 1.80 Å resolution structure PDB Code 1TPH (Zhang et al. 1994; Berman et al. 2000) of the TIM-phosphoglycolohydroxamate complex. Theoretical titration functions were obtained for the biologically active dimer from which the coordinates for the inhibitor were removed.

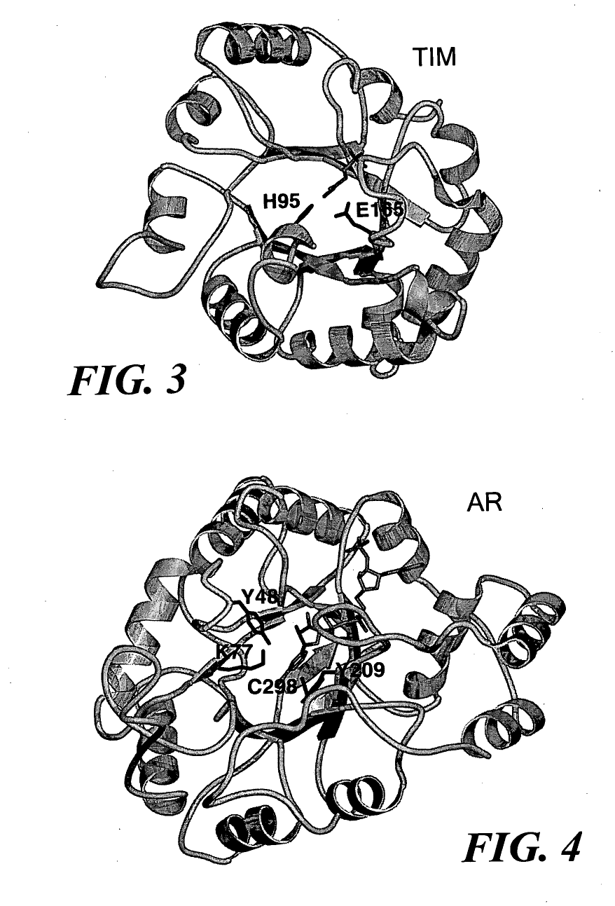

The observations of the theoretical titration functions for all of the ionizable residues of TIM indicated that four residues have curves with highly perturbed shapes: His95, Glu165, Lys112 and Tyr164. FIG. 2 shows the predicted mean net charge as a function of pH for the eight histidine residues in the A-chain. Predicted titration functions for the B chain (not shown) were similar to the A chain. FIG. 3 shows the location of His95, Glu165 and Tyr164 in the active site of TIM.

The theoretical titration functions for most of the histidine residues depicted in FIG. 2 have the typical shape. However, it was observed that His95 has a strikingly different predicted titration function. Not only was the pKₐ of the conjugate acid downshifted to 3.2, but also the shape of the curve was perturbed; in particular, the curve for His95 has a flat region where the mean net charge stays nearly constant over a few pH units (about pH −2.0 to +1.5). Thus, His95 was predicted to stay partially protonated over a wide pH range. This change in shape of the titration curve was the most significant difference between His95 and the other histidine residues. The predicted mean net charge for His95 is 0.10, 0.03 and 0.00 for pH 5.0, 6.0 and 7.0 respectively, consistent with experimental data, as discussed below. Similarly the predicted titration curve for Glu165 was different from the other glutamates. Again there was an unusual, nearly flat region in the curve (at about pH 1.0 to 4.0) and the residue is predicted to be partially protonated over an uncommonly wide pH range, with a downshifted pKₐ of −0.5. A third residue, Lys112, was found to have a flat region of partial protonation in the pH range 11-14, with an upshifted pKₐ of about 16.5 for its conjugate acid. A fourth residue, Tyr164, was found to have a nearly flat region of partial ionization in the theoretical titration curve (at about pH 13.0-16.0). It was predicted to remain uncharged up to pH 13 with predicted charges of −0.01, −0.03, and −0.10 at pH 13.0, 15.0, and 17.0, respectively and a calculated pKₐ of 18.2. Thus it was predicted to have an unusually high pKₐ. Furthermore the shape of the predicted titration curve was noticeably different from those of the other tyrosine residues.

Three of the four residues with perturbed titration curves are in close spatial proximity: His95, Glu165 and Tyr164. This region of physical space correlates with biochemical evidence for the location of the active site. Structural evidence suggests (Zhang et al. 1994) that the two residues that are active in acid/base catalysis are Glu165 and His95. Earlier affinity-labeling experiments established the side chain of Glu165 as the catalytic base (Coulson et al. 1970; Waley et al. 1970; Hartman 1970). Spectroscopic evidence suggested that an electrophilic residue was involved in the catalysis (Komives et al. 1991) and the x-ray crystal structure revealed His95 as the likely electrophile (Zhang et al. 1994). ¹⁵N NMR spectra show that the imidazole ring of His95 is substantially uncharged over the pH range 4.9-9.9, implying that the pKₐ is less than 4.5 (Lodi et al. 1991). The predicted mean net charges and calculated pKₐ of 3.2 for His95 were consistent with the ¹⁵N NMR data. Tyr164 is located right next to the active site. The x-ray crystal structure indicates that it is pointing away from the precise location where the substrate is believed to bind (Zhang et al. 1994), although this does not necessarily preclude involvement in catalysis. Lys112 is physically well removed from the active site and is considered to be a false positive.

The above analysis pertains to four residues originally identified by human observers as having anomalously shaped titration curves. A subsequent statistical analysis of the P values (as defined, above, by Equation 4) and the moments of the function f (Equation 7) identified a slightly different set of outliers: His95, Glu 165, C126, and Y164. These four residues form a single cluster. Again, His95 and Glu165 are catalytic residues while C126 and Y164 are second shell residues. The important point here is that, while the list of positive residues is slightly different depending on the type of analysis used, the catalytic site and the important catalytic residues for this known active site are identified by the method of the invention.

### EXAMPLE II

### Aldose Reductase (AR)

AR (Harrison et al. 1994; Wilson et al. 1992; Bohren et al. 1994) is an NADPH-dependent enzyme that catalyzes the reduction of the aldehyde group in an aldose to the corresponding alcohol. Calculations were performed on the biologically active monomer. The 1.76 Å resolution x-ray crystal structure 2ACS (Harrison et al. 1994) of human aldose reductase from the Protein Data Bank (Berman et al. 2000) was utilized. The structure 2ACS contains the nicotinamide cofactor and a citrate ion; the calculation was performed on the protein alone without the cofactor or ion. FIGS. 3 and 4 show the relationship between the structures of TIM and AR.

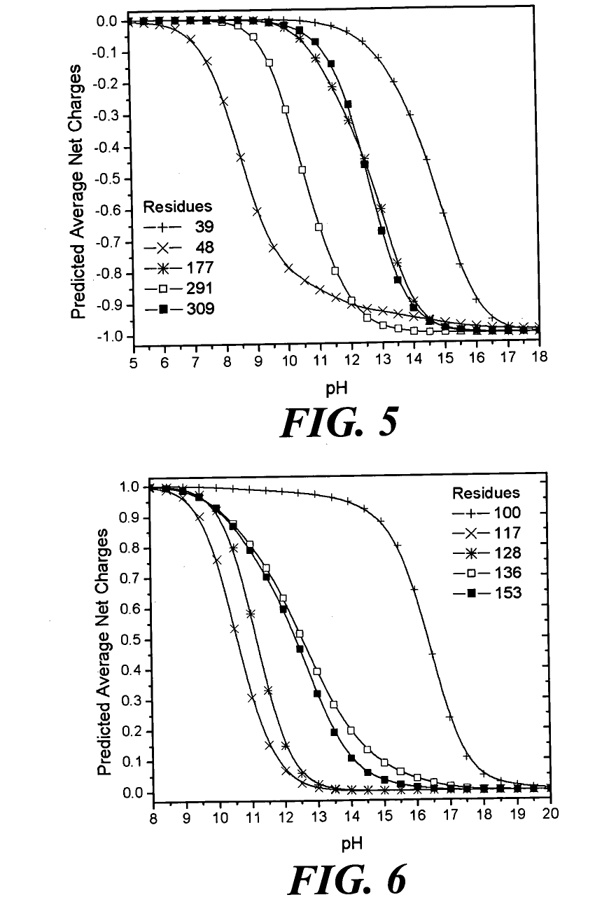

The theoretical titration functions for AR reveal seven residues with unusual curves: Tyr48, Cys298, Glu185, Lys21, Lys77, Tyr107 and Tyr209. FIG. 5 shows the predicted mean net charge as a function of pH for five selected tyrosine residues that show typical curves but do not obscure the curve for Tyr48. Tyr48 is predicted to have an extended region of partial protonation (i.e. non-integer mean net charge) in the pH range 12.5-17.5. The curve for Tyr48 is nearly flat from about pH 12.0 to 15.0, with a long tail on the higher pH end that crosses over curves of residues with higher pKₐ. The pKₐ of Tyr48 is calculated to be 8.4, at least a few pH units lower than the other tyrosine residues shown. The low pKₐ and the flat region at the higher pH side of the curve make Tyr48 partially protonated over the remarkably wide pH range of 5.0 through 17.5. The other tyrosine residues in FIG. 5 have sigmoidal, or nearly sigmoidal, theoretical titration curves shapes, which are characteristic of an ordinary Henderson-Hasselbalch residue. It is also observed that Cys298 has a wider range of partial protonation than the other cysteine residues and a flat region in the pH range 14-16. Its calculated pKₐ of 10.2 is similar to most of the other cysteine residues in this protein, although a little higher than typically observed for a thiol group free in solution. The calculated titration curve for Glu185 exhibits a nearly flat region in the pH range 0-3; the calculated pKₐ of −1.7 is significantly downshifted from the other glutamates. Calculated mean net charges for Glu185 are −0.94, −0.97, −0.98, and −0.99 for pH 0.0, 1.0, 2.0, and 3.0, respectively. The titration curve for Lys21 has a flat region of non-integer net charge in the pH range 8-11, with a predicted pKₐ of 13.4 for its conjugate acid. Lys77 has an extended flat region of partial protonation in the pH range 9-17, with a heavily upshifted predicted pKₐ of 19.6 for the conjugate acid. The predicted curve for Tyr107 exhibits a flat region in the pH range 11-15 with non-integer mean net charge; the pKₐ is predicted to be upshifted to 17.0. Finally, Tyr209 was found to have an extended flat region of partial protonation in the pH range 9.5-15.5, with a very upshifted predicted pKₐ of 17.9. An eighth residue, His110, has an extended region of partial protonation but we did not count it in the original visual analysis because the differences in its titration curve are not as apparent as they are for the seven residues discussed above. Statistical analysis later identified His110 as an outlier, as described below.

Again, the majority of the residues with positive results are close in space and the active site location indicated by the theoretical titration functions is consistent with biochemical data. The 1992 x-ray crystal structure at 1.65 Å resolution suggested that Tyr48, His110 and Cys298 are the catalytically active residues (Wilson et al. 1992). Site-directed mutagenesis experiments showed that the Y48F mutant has no activity and the H110Q and H110 Å mutants lose activity by 1*10³-2*10⁴ fold (Bohren et al. 1994); thus it was concluded that Tyr48 is the proton donor and His110 is involved in the catalysis. Reference 30 also reports pH profiles of kinetic data and from them infers that the pKₐ of Tyr48 in the wild type enzyme is 8.4, with which our calculated value is in good agreement. Four of the seven residues with positive results, Tyr48, Cys298, Lys77 and Tyr209, were all identified as active site residues from structural and biochemical data (Harrison et al. 1994). Two more of the seven, Glu185 and Lys21, are located right next to active site residues: Lys21 is just behind active site residue Trp20 and also next to the cofactor; Glu185 is located just behind active site residues Tyr209 and Cys298. Tyr107 is located behind active site residue Lys77, but is far enough away from the catalytic site that we consider it to be a false positive.

The above analysis pertains to the residues originally identified as positive by human observers. Subsequently, statistical analysis added His110, a known catalytic residue, to the list. His110 is in physical proximity to residues in the active site cluster and, therefore, is a member of that cluster. Thus, as will be shown in more detail in some of the following examples below, statistical analysis provides a more complete list of important residues.

### EXAMPLE III

Phosphomannose Isomerase (PMI)

PMI (Cleasby et al. 1996; Gracy et al. 1968; Wells et al. 1994; Malaisee-Lagae et al. 1989) catalyzes the reversible interconversion of mannose-6-phosphate and fructose-6-phosphate. PMI is a metal-dependent enzyme containing one atom of zinc per protein molecule. The 1.70 Å resolution x-ray crystal structure of 1PMI (Cleasby et al. 1996), the enzyme from Candida albicans, was used in the calculations here. The zinc ion was included in the calculation.

Upon examination of the theoretical titration functions for PMI, we find four residues with perturbed curves that possess a flat or nearly flat region: His135, Lys100, Lys136, and Tyr287. The titration curve for another residue, Glu294, has an obviously perturbed curve in that its slope is considerably less steep than that of the other glutamates. It is predicted to be partially protonated over the very wide range of about pH −2.0 through 7.0 with a pKₐ of 2.5. Specifically, it lacks a flat region of partial protonation but instead has an unusually shallow, nearly constant, slope over a wide pH range. For present purposes, we are interpreting the curves in conservative fashion and are excluding Glu294 from the positive list.

FIG. 6 shows the predicted mean net charge as a function of pH for five selected lysine residues in PMI. The lysine residues were selected to show some with typical curves (Lys117 and Lys128), one with a slightly perturbed slope (Lys153), and the two curves with unusual shapes (Lys100 and Lys136). Lys100 exhibits a flat region of non-integer mean net charge at about pH 11-14; its conjugate acid is predicted to have a high pKₐ of 16.4. Lys136 has a more typical pKₐ (of 12.5 for the conjugate acid) but it has a tail on the high pH end of its titration curve, with a flat region at about pH 16.0-17.5. Also His135 has a flat region of partial protonation in the pH range 1-4; its predicted pKₐ of 5.8 is a typical value for the conjugate acid of a histidine residue. Tyr287 has a flat region of non-integer mean net charge in the pH range 14.5-17.0 and has a very unusually high pKₐ (>19).

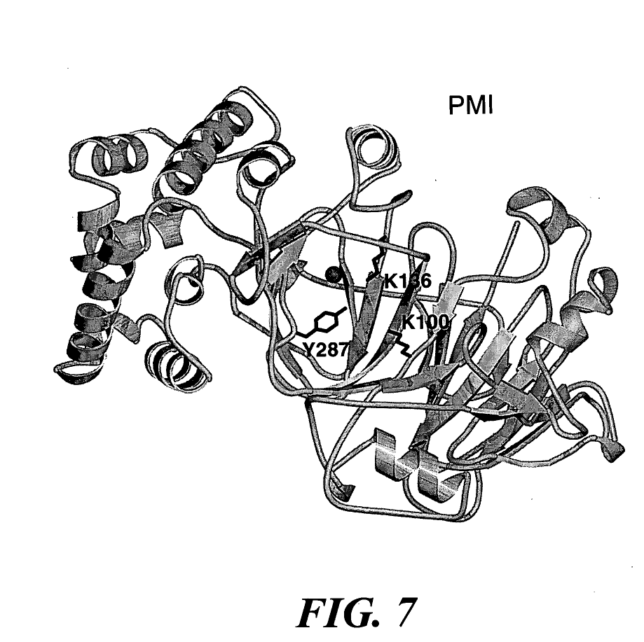

The precise mechanism of action of PMI is not yet known. However, because it is a metalloenzyme and the metal ion is essential for activity (Gracy et al. 1968), the active site is presumed to be located in an observed cleft near the zinc ion. The structural data of reference 32, together with labeling studies (Wells et al. 1994), identify six ionizable active site residues that are not involved in coordination of the zinc ion: Arg304, Glu48, Glu294, Lys100, Lys136, Tyr287. FIG. 7 shows the structure and some of the presumed active site residues for PMI. Experimental evidence supports a proton transfer mechanism via a cis ene-diol intermediate (Malaisse-Lagae et al. 1989), rather than by a hydride shift as for xylose isomerase (Lavie et al. 1994; Carrell et al. 1994).

Therefore, of the four residues with a positive result, all are in close proximity and three residues, Lys100, Lys136 and Tyr287, are in the presumed active site. His135 is located just behind the presumed active site residue Lys136 and, therefore, is a second shell residue.

Application of THEMATICS to theoretical model structures (EXAMPLES IV-VII). In Examples IV-VII, we start with the primary sequence and then build a theoretical model structure. Then THEMATICS is applied on the theoretical model structure.

### EXAMPLE IV

### Triosephosphate Isomerase (TIM) Orthologs

The first of the four structures homologous to the chicken triosephosphate isomerase (TIM) structure 1TPH is built from the sequence for Schistosoma japonicum (Liu et al. 2003) with 60% sequence identity in the pair wise alignment and 0.16 Å RMSD value for the model structure. The second model is determined for the sequence for Enterococcus faecalis (Paulsen et al. 2003) with 40.2% sequence identity, resulting in a 0.29 Å RMSD value with the template structure. The third model is built from the sequence of Bartonella henselae with 38.7% identity and RMSD value of 0.31 Å, while the last model is built from the sequence of Mycoplasma genitalium (Frase et al. 1995) with 33% identity and RMSD value of 1.73 Å. These structures are all obtained with MODELLER and are summarized in Table 2 (shown below).

Table 2 gives the THEMATICS result for the active site cluster for each template structure and the orthologous model structures. Known active site residues are shown in boldface and second shell residues (those immediately adjacent to active site residues but not considered to be in the active site) are underlined.

For the TIM structure from chicken (1TPH), four neighboring residues with anomalous titration behaviour are identified as the active site cluster. Two of these residues, H95 and E165, are well established by experiment as catalytically active residues (Lodi et al. 1991; Zhang et al. 1994) Two other residues, C126 and Y164, are located in the active site cleft but any possible catalytic role for these residues has not been investigated experimentally. Upon alignment of the sequences and superposition of the structures, it is confirmed that all four of these residues are conserved, both in the sequence and in the spatial arrangement of the active site cleft, in all of the four model structures. Sequence alignment across a wider range of species again reveals high conservation of all four of these residues.

For all four of the model structures, THEMATICS finds the active site. THEMATICS identifies (by pronounced perturbed shape of the predicted titration curves) the two catalytic residues H95 and E165, plus C126, in all four of the model structures. For two of the four models, S. japonicum and B. hensalae, Y164 also exhibits perturbed titration behaviour. Y164 does not show significant perturbed titration behaviour in the model structures for E. faecalis and M. genitalium. Thus THEMATICS identifies the correct active site cluster for all of the model structures, but Y164 is not always included in the predicted active site cluster.

### EXAMPLE V

### 6-Hydroxymethyl-7,8-dihydropterin pyrophosphokinase (HPPK) orthologs

6-Hydroxymethyl-7, 8-dihydropterin pyrophosphokinase, HPPK, is a monomeric pyrophosphate transferase with α-β plaits topology. Its crystal structure for E. coli (PDB code 1HKA) was obtained from the protein data bank with 1.5 A resolution (Xiao et al. 1999). Four homologous models to the E. coli structure 1HKA are built using the MODELLER software from the sequences for the following organisms: Vibrio vulnificus (Rhee et al. 2002) (with 63% sequence identity with E. coli and 0.34 Å RMSD), Vibrio parahaemolyticus (Makino et al. 2003) (with 57% sequence identity and 0.22 Å RMSD), Pseudomonas aeruginosa (Stover et al. 2000) (with 51% sequence identity and 0.36 Å RMSD) and Pseudomonas putida (Nelson et al. 2002) (with 48% sequence identity and 0.50 ÅRMSD).

The predicted titration curves for residues D95, D97 and H115 in the template E. coli structure do not show the usual sigmoidal behaviour and are identified as the active site cluster by statistical analysis. All three of these residues have been identified as active site residues in an experimentally determined inhibitor structure (Stammers et al. 1999). All of them are conserved across the four species for which the model structures are built and are also generally well conserved across bacterial kinases. When the four model structures are overlaid onto the template E. coli structure, the positions of these residues are conserved in the active site pocket with similar orientations.

For the HPPK case, THEMATICS identifies the same cluster for all four of the model structures as for the E. coli template structure (see Table 2, shown below).

### EXAMPLE VI

### Aspartate Aminotransferase (AspAT) orthologs

The structure of the pyridoxamine 5′-phosphate (vitamin B6) dependent enzyme Aspartate aminotransferase (AspAT) from E. coli at 2.2 Å resolution (PDB code 1AMR) (Miyahara et al. 1994) is used as the template. Its fold is a unique aminotransferase fold. AspAT is active as a homodimer and the calculations are performed on the dimer structure.

Using MODELLER software (Fiser et al. 2000), four model structures homologous to the AspAT template from E. coli are constructed from the sequences for the following organisms: Vibrio cholera (Heidelberg et al. 2000) (with 62% pairwise identity and 1.52 Å RMSD), Oryza sativa (Sasaki et al. 2001) (with 44% identity and 0.64 Å RMSD), and Neiserria meningitides (Tetelin et al. 2000) (41% identity and 1.28 Å RMSD), and Clostridium perfringens (22% identity and 3.67 Å RMSD).

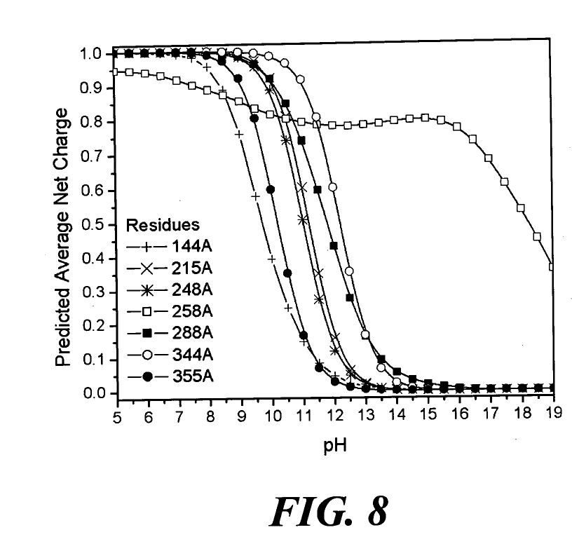

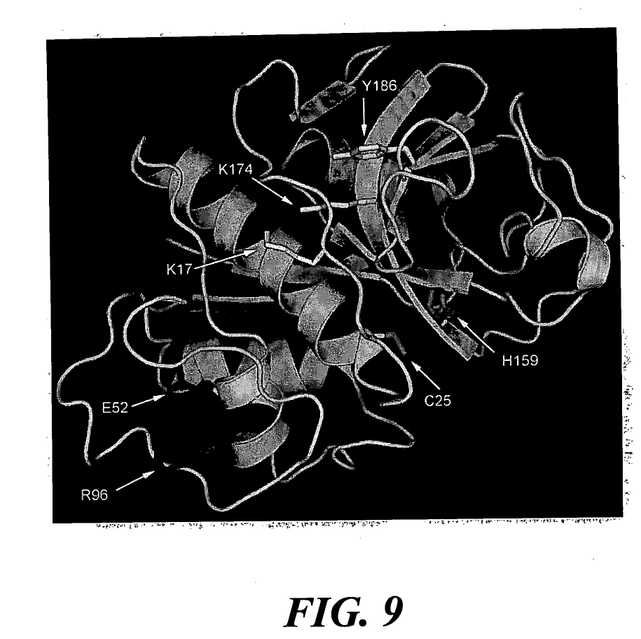

FIG. 8 illustrates the predicted titration curves for some of the lysine residues of the template structure for E. coli. Predicted mean net charge as a function of pH is shown for all of the lysine residues in the sequence between 144 and 355 (inclusive) of the A chain of the homodimer. The titration curves are given for K144 (+), K215 (×), K248 (*), K258 (□), K288 (▪), K344 (◯), and K355 (●). Note the elongated, highly non-sigmoidal shape of the catalytic lysine residue K258. The other lysine residues all have sigmoidal or nearly sigmoidal shape, with the characteristic sharp fall-off in charge in the region where the charge is approximately equal to one-half.

For the template E. coli structure, THEMATICS finds a cluster of residues with perturbed titration behaviour, consisting of the following: H189, Y225, K258, R266, C191 and C192. It has been established experimentally that K258 is the catalytic base that initiates transamination, that H189, Y225 and R266 are also in the active site pocket, and that nearby residues outside the active site, such as C191, also play a role in the catalytic activity (Miyahara et al. 1994; Jeffery et al. 1998; Jeffery et al. 2000; Mizuguchi 2001). When sequential alignment is performed on all of the model sequences and the E. coli sequence, the identified residues are all conserved, except that C191 and C192 are not present in Clostridium perfringens. Superposition of the model structures onto the template structure reveal that the identified residues are located in the same region of the protein with similar orientations.

For all four models and for the template, THEMATICS finds the active site cluster, although the list of identified residues is a little different for each species for the AspAT case (see Table 2, shown below).

TABLE 2
Summary of Orthologous Model Structures and Results

| Enzyme | Species | % Identity | RMSD | THEMATICS Result |
|---|---|---|---|---|
| TIM | S. japonicum | 60% | 0.16 | [H95, E165, C126, Y164] |
| | E. faecalis | 40% | 0.29 | [H95, E165, C126] |
| | B. hensalae | 39% | 0.31 | [H95, E165, C126, Y164] |
| | M. genitalium | 33% | 1.73 | [H95, E165, C126] |
| | Template structure: | | | [H95, E165, C126, Y164] |
| | Chicken (1TPH) | | | |
| HPPK | V. vulnificus | 63% | 0.34 | [D95, D97, H115] |
| | V. parahaemolyticus | 57% | 0.22 | [D95, D97, H115] |
| | Ps. aeruginosa | 51% | 0.36 | [D95, D97, H115] |
| | Ps. putida | 48% | 0.50 | [D95, D97, H115] |
| | Template structure: | | | [D95, D97, H115] |
| | E. coli (1HKA) | | | |
| AspAT | V. cholerae | 62% | 1.52 | [H189, Y225, K258, C191, C192] |
| | Oryza sativa | 44% | 0.64 | [H189, Y225, K258, R266, C191, |
| | | | | Y295] |
| | N. meningitides | 41% | 1.28 | [H189, Y225, K258, C191, C192] |
| | C. perfringens | 22% | 3.67 | [H189, Y225, K258, R266] |
| | Template structure: | | | [H189, Y225, K258, R266, C191, |
| | E. coli (1AMR) | | | C192, Y256] |

For each model structure, % pairwise identity with the template and the RMSD value in Å are given.
THEMATICS results for the active site cluster are given with known active site residues shown in boldface and the second shell residues underlined.
Sequence numbers for the models are adjusted to match those of the template structures.

### EXAMPLE VII

Human Homologues of Aldose Reductase

Aldose reductase is an NADH-dependent enzyme that catalyses the reduction of the aldehyde group in an aldose to an alcohol. It has the α/β barrel (“TIM barrel”) fold and is active as a monomer. The x-ray crystal structure data are obtained from the protein data bank (PDB code 2ACS) with 1.76 Å resolution (Harrison et al. 1994).

Homologous structures to human aldose reductase are built using SWISS-MODEL (Schwede et al. 2003). Model structures are constructed using homologous protein sequences from the human and the template structure 2ACS for aldose reductase. These model protein structures all have the same fold as aldose reductase but they perform different functions and catalyse different reactions. The following model protein structures have been constructed: aldehyde reductase (Wermuth et al. 1987), (with 50% pair wise identity), bile acid binding protein (Stolz et al. 1993) (with 48% identity), 3-oxo-5-beta steroid dehydrogenase (Kondo et al. 1994) (50% identity), and chlordecone reductase (Qin et al. 1993) (48% identity). The x-ray structures for aldehyde reductase (2ALR) and bile acid binding protein (1IHI) are available from the PDB and were used to check our results for the model structures.

THEMATICS computation on the template aldose reductase structure identifies by statistical analysis the following cluster of residues as important: C298, H110, K77, Y48, Y209, E185 and K21. C298, H110, K77, Y48, Y209 are known active site residues while E185 and K21 are just behind the active site in the second shell surrounding the reacting substrate (Harrison et al. 1994). Results are summarized in Table 3. For the models, the residues that occupy equivalent positions in the structure are vertically aligned in Table 3. Only the residues that are predicted to have perturbed titration behaviour are shown. As these enzymes have different functions, not all of the residues in the THEMATICS active site cluster are conserved, although K77, Y48 and E185 are conserved.

THEMATICS correctly locates the active site cluster for these human homologues of aldose reductase, in spite of some sequence variability in the active site and in spite of differences in function. Note that residues in equivalent positions are sometimes identified by THEMATICS even when there is amino acid substitution at that location.

It is also instructive to compare the THEMATICS results for the model structures of aldehyde reductase and bile acid binding protein with the results for the experimentally determined x-ray crystal structures. For aldehyde reductase, five out of the seven residues identified for the model structure are also identified for the crystal structure (H112, K79, Y49, Y209 and E185); two of the seven (K22 and Y297) are identified for the model structure but not for the crystal structure. For bile acid binding protein, the model structure and the crystal structure both identify five active site cluster residues: E117, K84, Y55, Y216, and E192. K27 is identified for the model structure but not for the crystal structure. Both structures give negative results for R304 (which occupies the position corresponding to C298 in aldose reductase).

TABLE 3
Human Homologues of Aldose Reductase

| Enzyme/% Identity | THEMATICS result | | | | | | |
|---|---|---|---|---|---|---|---|
| Template: | | | | | | | |
| Aldose reductase | [C298, | H110, | K77, | Y48, | Y209, | E185, | K21] |
| Models: | | | | | | | |
| Aldehyde reductase/ | Y297 | H112 | K79 | Y49 | Y209 | E185 | K22 |
| 50% | | | | | | | |
| Bile acid binding | np¹ | E117 | K84 | Y55 | Y216 | E192 | K27 |
| protein/48% | | | | | | | |
| 3-oxo-5-beta steroid | np² | H113 | K80 | Y51 | Y212 | E188 | np³ |
| dehydrogenase/50% | | | | | | | |
| Chlordecone | Y304 | H116 | K83 | Y54 | H215 | E191 | np⁴ |
| reductase/48% | | | | | | | |

THEMATICS results are given for human aldose reductase and four human homologues with different chemical functions. Residues occupying the same position in the structure are aligned vertically in the table. In the template, known active site residues are shown in boldface; second shell residues are underlined. Only perturbed residues (those identified by THEMATICS) are shown.
np = the residue in that position is not perturbed and is not identified by THEMATICS;
np¹ = R304;
np² = Y301;
np³ = E24;
np⁴ = E27

### EXAMPLE VIII

Use of THEMATICS Finds Two Positive Clusters in Papain

Papain is a cysteine protease from papaya with a hydrolase fold. The monomeric structure 1PIP [Yamamoto et al. 1992] at 1.7 Å resolution was used as input.

Papain is one of the relatively unusual cases in which THEMATICS has found two positive clusters. One of these clusters, [C25, H159], is the known protease active site. The cysteine C25 acts as a nucleophile to attack the carbon atom of the scissile bond. H159 acts as a proton shuttle. The function for the other cluster [K17, K174, Y186] is currently unknown. In addition, THEMATICS finds two isolated residues, E52 and R96, that have anomalous curve shapes. FIG. 7 shows a ribbon diagram of papain with the side chains of the active site cluster [C25, H159] shown in red, the second cluster [K17, K174, Y186] shown in yellow, and the two isolated residues E52 and R96 shown in blue.

A sequence alignment was performed for plant cysteine proteases from alder, Arabidopsis thaliana, barley, ice plant, kiwi, rice, and sweet potato and compared with papain. Sequence searches were preformed using WU-Blast2 http://blast.wustl.edu/ [Gish et al. 1993] and sequence alignments were performed with ClustalW http://www.ebi.ac.uk/clustalw [Thompson et al. 1994]. The overall sequence identity scores for these proteins with papain are in the range 45-51%. Table 4 (shown below) shows the alignment results for all of the residues determined to be anomalous in papain by THEMATICS. Note how all five residues in the two positive clusters are perfectly conserved across the species, but the two isolated anomalous residues, E52 and R96, are not perfectly conserved. E52 is conserved in all but two of the species; R96 is not conserved.

TABLE 4
Sequence alignment results of the anomalous residues
for papain across similar plant proteases using THEMATICS.
(Percent identity with papain is shown in parentheses.)

| Species | [C25, | H159] | [K17, | K174, | Y186] | [E52] | [R96] |
|---|---|---|---|---|---|---|---|
| Papaya | C | H | K | K | Y | E | R |
| Alder (48%) | C | H | K | K | Y | E | N |
| Arabidopsis | C | H | K | K | Y | Q | S |
| (49%) | | | | | | | |
| Barley (49%) | C | H | K | K | Y | E | N |
| Ice plant (45%) | C | H | K | K | Y | Q | K |
| Kiwi (49%) | C | H | K | K | Y | E | N |
| Rice (51%) | C | H | K | K | Y | E | R |
| Sweet potato | C | H | K | K | Y | E | K |
| (49%) | | | | | | | |

### EXAMPLE IX

### Characterization of Protein Function Applying THEMATICS to Serine Proteases

The specific serine protease Kex2 was compared to the structurally related non-specific serine protease subtilisin Carlsberg. These two proteins have identical catalytic residues but one has specificity determinants that the other protein lacks. THEMATTICS identifies the catalytic residues for both specific and non-specific proteases and also identifies the recognition residues for a specific protease. The ability to identify sites that govern recognition opens the door to better understanding of specificity and to the design of highly specific inhibitors.

The active site of an enzyme in the serine protease family possesses the “catalytic triad,” an acid, a histidine, and a serine, where the acid is an aspartate or glutamate side chain, the histidine acts as a catalytic base, and the serine serves as a nucleophile [Holmquist 2000; Di Cera et al. 2003]. This catalytic triad performs hydrolysis on peptide bonds. In the case of a specific protease, the protein recognizes the side chain of one or more residues adjacent to the peptide bond to be hydrolyzed. The residue that provides the carbonyl carbon atom of the reactive peptide bond is labeled as P₁. The pocket in the protease that recognizes the side chain of the P₁ residue is called the S₁ pocket. Similarly, the next residue to the N-terminal side of the substrate protein is labeled as P₂ and it is recognized by the S₂ site of the protease, and so on.

Kex2 (kexin; E.C. 3.4.21.61) is a calcium dependent transmembrane protease found in the yeast Saccharomyces cerevisiae. The name derives from the killer phenotype of Kex2 mutant cells. In Saccharomyces cerevisiae, Kex2 is required for the production and secretion of mature alpha-mating factor and killer toxin, among other activities. Proteolysis occurs at paired basic residues.

Kex2 is the prototype of a large family of eukaryotin pro-protein processing proteases that includes furin, the PC's, and PACE4 in mammals. This family of proteases is responsible for the processing of neuropeptides and peptide hormones, proinsulin, coagulation factors and many growth hormones and their receptors, Alzheimer-related secretases and cancer-associated extracellular matrix proteinases. In addition, furin is known to function in embryogenesis and homeostasis, and is required for the activation of many bacterial toxin precursors and virus envelope glycoproteins.

Kex2 specifically cleaves peptide bonds where P₁ and P₂ are the basic residues arginine and lysine. (Kex2 cleaves preferentially after RR and RK.) Kex2 belongs to a family of proteases structurally related to the subtilisins. However subtilisin is a very non-specific protease the structural similarity does not account for the unusual specificity observed for Kex2. Inhibited structures of the catalytic and P domains of Kex2 [Holyoak et al. 2003] and furin [Henrich et al. 2003] have been determined, and show the source of the calcium dependence and the nature of the high specificity.

Subtilisin is mechanistically and structurally related to Kex2. However subtilisin is non-specific and cleaves peptide bonds in proteins with little regard for the nature of the surrounding residues, except for a small preference for hydrophobic residues. Subtilisin is used commercially in laundry detergents to degrade proteinaceous material into small, soluble fragments.

THEMATICS calculations were performed on the 2.4 Å resolution structure of Kex2 [Holyoak et al. 2003] from yeast, Protein Data Bank (PDB) [Berman et al. 2000] code 10T5 and on the 1.2 Å resolution structure [Bode et al. 1987] of subtilisin Carlsberg from Bacillus subtilis, 1CSE. Table 5 shows the results of these calculations for the two featured proteins plus some additional enzymes chosen to highlight different types of recognition. THEMATICS positive residues for these examples are known to be involved in catalysis and recognition.

THEMATICS positive residues for Kex2 were found to be: [D175, D176, D210, D211, E220, H213, H381, Y212], [D277, D320, E350], [D184], [Y308], where residues that are clustered together in coordinate space are shown together in square brackets and where known catalytic residues are shown in boldface and known recognition residues are shown in italics. The first and largest cluster includes the ionizable catalytic residues D175 and H213. This same cluster also includes the S2 recognition residues, D176, D210 and D211. The second cluster contains the S1 recognition residues D277, D320 and E350. D184 and Y308 are each isolated in space and their functional significance is currently unknown.

THEMATICS positive residues for subtilisin were found to be [D32, H64], the acid and histidine residues of the known catalytic triad, and D41 (presumed to be an isolated false positive). No residues, analogous to those in the first two clusters for Kex2, indicative of specificity were found.

Table 5 also summarizes THEMATICS results for some additional enzymes. The last two enzymes in the Table are active as dimers and the calculations for these were performed on the dimer structure. In all of these enzymes, the method finds at least one residue involved in catalysis and, except for the non-specific subtilisin, at least one residue involved in recognition. Overall, these enzymes represent a variety of different kinds of chemistry and topology and different types of recognition. Kex2 utilizes the ionizable side chains of the acidic residues aspartate and glutamate to recognize arginine in the S1 pocket and either arginine or lysine in the S2 pocket; THEMATICS identifies residues in both of these pockets. For arginine kinase [Zhou et al. 1998], THEMATICS identifies a set of arginine residues that recognize a phosphate group. Creatine amidinohydrolase [Coll et al. 1990] has two glutamate side chains that recognize a guanidinium group of the substrate, and these glutamate residues are found to be positive by THEMATICS. THEMATICS also identifies as positive the residues in the site where the halogen is recognized in L-2-haloacid dehalogenase [Li et al. 1998; Ridder et al. 1999].

TABLE 5
THEMATICS results of positive residues that are known
catalytic residues (bold) or known recognition residues
(italicized), with residues in physical proximity in the protein
shown together in square brackets. Prime indicates a
residue from another subunit in a dimer.

| PDB ID/ | Name | Chemistry | Topology |
|---|---|---|---|
| 1OT5 | Kex2 | serine protease | subtilisin + |
| | | | jelly roll |

catalytic residues: D175, H213
basic side chain recognition: D277, D320, E350, D176, D210, D211
all positives: [D175, D176, D210, D211, E220, H213, H381, Y212], [D277,
D320, E350], [D184], [Y308]

| | | | |
|---|---|---|---|
| 1CSE | Subtilisin Carlsberg | serine protease | subtilisin |

catalytic residues: D32, H64
all positives: [D32, H64], [D41]

| | | | |
|---|---|---|---|
| 1BG0 | Arginine kinase | phosphate transfer | creatine |
| | | | kinase |

catalytic residues: E225
phosphate recognition: R126, R229, R280, R309
all positives: [R126, R229, D226, E224, E225], [R124, R280, R309, R330],
[C127], [E335], [H185], [Y134], [Y145]

| | | | |
|---|---|---|---|
| 1CHM | Creatine amidinohydrolase | C—N bond | unique |
| | | hydrolysis | |

catalytic residues: H232
guanidinium recognition: E262, E358
all positives: [R64′, R335, D83′, E262, E358, H232, H324, H376], [D217,
H331′, Y48], [E124], [Y54], [Y257]

| | | | |
|---|---|---|---|
| 1QQ5 | L-2-Haloacid dehalogenase | dehalogenation | unique |

catalytic residues: D8, D176, K147
recognition residues for halide: R39
all positives: [R39, D8, D176, K147, Y153], [R217′, E44, K41, Y45, Y68], [Y223]

### EXAMPLE X

### Application of Statistical Analysis to Predicted Titration Curves for HPPK

The enzyme HPPK (6-Hydroxymethyl-7,8-dihydropterin pyrophosphokinase), a bacterial kinase, is discussed above in Example V. We illustrate how statistical analysis can be used to select the residues with anomalous titration behavior, using the complete set of predicted titration curves C(pH) for the HPPK structure from the PDB (1HKA) for E. coli. We analyze the third and fourth moments of the first derivative function of the titration curves for each of the ionizable residues. In this example, we analyze all of the ionizable residues together. (In a variation of this procedure, we compare only residues of the same type, so that arginines are analyzed separately, as are aspartates, etc.) The absolute value of the third moment and the fourth moment are two of the preferred measures to determine the degree of deviation from normal behavior. Table 6 shows the residues with the highest absolute value of the third moment. This enzyme has 45 ionizable residues; the ten with the highest third moment magnitudes are shown. Residues are listed in rank order, with the highest value first. For each residue, a label is given, indicating whether the residue is known to be in the active site or in the second shell. The label “No” indicates that it has no known functional role. Residues above the solid line in the table have an absolute value of the third moment that is more than one standard deviation away from the mean value (Mean=0.31; σ=0.75). Notice that the active site residues, shown in boldface, tend to be at the top of the list. Second shell residues are shown in italics. Notice that, if the absolute value of the third moment magnitude is used as the sole criterion for selection and the cut-off is set at greater than one standard deviation from the mean, this statistical procedure finds the active site cluster [D97, H115], consisting of the two known catalytic residues, and one isolated false positive, E77. However, this procedure misses D95, which is located in the active site pocket and might play a role in catalysis.

TABLE 6
HPPK - Residues with Highest Absolute
Value of the Third Moment

| | | Absolute Value |
|---|---|---|
| Residue | Label | of Third Moment |
| Asp97 | Active site | 4.63 |
| His115 | Active site | 1.75 |
| Glu77 | No | 1.15 |
| Arg84 | No | 0.65 |
| Asp95 | Active site | 0.59 |
| Tyr53 | No | 0.57 |
| Tyr40 | No | 0.33 |
| R121 | No | 0.30 |
| D139 | No | 0.23 |
| E67 | No | 0.23 |

Table 7 shows the ten residues with the highest values for the fourth moment. Residues are listed in rank order, with the highest fourth moment first. Residues above the solid line in the table have an absolute value of the fourth moment that is greater than one standard deviation away from the mean value (Mean=3.6; σ=4.1). Again, the active site residues are at the top of the list.

TABLE 7
Residues with Highest Fourth Moment

| Residue | Label | Fourth Moment |
|---|---|---|
| Asp97 | Active site | 25.9 |
| Asp95 | Active site | 10.9 |
| His115 | Active site | 9.8 |
| Glu77 | No | 9.6 |
| Arg84 | No | 4.9 |
| Glu68 | No | 4.3 |
| Tyr53 | No | 4.2 |
| His72 | No | 3.8 |
| Asp139 | No | 3.6 |
| Glu141 | No | 3.4 |

If the fourth moment is used as the sole criterion for selection and the cut-off is set at greater than one standard deviation from the mean, this statistical procedure finds the active site cluster [D95, D97, H115] and one isolated false positive, E77.

This example illustrates how statistical analysis of the curve features can be used to identify THEMATICS positive residues and THEMATICS positive clusters. A number of variations are possible in the statistical analysis, a few of which are shown above, that lead to the correct prediction of active sites.

### EXAMPLE XI

### Application of Automated Classification to Predicted Titration Curves

In a neural network (NN) binary classifier, there are several input nodes, one per numerical feature, and one output node, whose value determines the class label predicted by the network. In order to allow the network to compute a nonlinear function of its input, there is also at least one layer of hidden nodes. For a NN classifier each node computes a weighted sum of its inputs and passes the result through a sigmoid function, which is designed to be a differentiable approximation to a threshold function. The weights in the network are variable parameters that are adjusted to fit the training data.

We have applied Neural Nets (NN) and Support Vector Machines (SVM) to classify the predicted titration curves of residues as either positive (perturbed) or negative (ordinary). In these methods, the classifier is first trained on a set training data where a human has already performed this classification. The machine is then given a previously unseen example to classify.

The training data come from the visual classification of titration curve shapes by a human observer. The observer classifies each titration curve as either as positive (perturbed in a manner indicative of active site involvement) versus negative (ordinary Henderson-Hasselbalch). Here are some major results of our machine learning so far using neural networks: The charge curves for 21 proteins were used in a neural net analysis for training and testing. These proteins are acetylcholine esterase, aldose reductase, HIV-1 protease, 6-Hydroxymethyl-7,8-dihydropterin pyrophosphokinase (HPPK), triosephosphate isomerase (TIM), papain, pepsin, glutamate racemase, adenosine kinase, ACC synthase (ACCS), alanine racemase, aspartate aminotransferase, phosphomannose isomerase (PMI), colicin E3, subtilisin Carlsberg, ketosteroid isomerase (KSI), phosphoglucose isomerase (PGI), chorismate mutase, apurinic apyrimidinic endonuclease (Apel), germin and galactose mutarotase. These proteins have a total of 2,512 ionizable residues.

These tests used networks having 16 input nodes and a single hidden layer with 4-6 nodes. In each case, classifiers were created using two different strategies, first using the positive training examples as given and then replicating the positive examples multiple times to make up for the fact that there are many more negatives than positives.

For these studies a separate network was trained for each ionizable residue type (Arg, Asp, Cys, Glu, His, Lys, Tyr). In each case, twenty proteins were used for training and one was held out for testing. The machine learning results for the test protein either duplicate the results of obtained by the human observer, or they find the positive residues of the observer plus something that the observer originally overlooked, thus outperforming the human observer.

One example where the automated classifier outperformed the human observer is triosephosphate isomerase (TIM). TIM was held out and training was performed on the remaining 20 proteins. When presented with the titration curve data for TIM, the automated classifier found Lys13 to be a positive residue, in addition to the residues found to be positive by the human observer and by statistical analysis. Lys13 is an experimentally confirmed active site residue (Zhang 1994) that was originally overlooked by the human observers. Thus, the automated classifier gives a more complete list of active site residues than the simple visual or statistical classification methods. Similar results were obtained using SVM's.

### EXAMPLE XII

### Characterization of Active Sites with THEMATICS—Application to Some Vitamin B6 Dependent Enzymes

Useful information is derived from a comparison of THEMATICS results for enzymes with functional similarities and differences. Here we compare results for three vitamin B6 (PLP) dependent enzymes, Alanine racemase (AlaR); D-amino acid aminotransferase (DaAT); and Aspartate aminotransferase (AspAT). These three proteins represent three different fold types and three independent evolutionary lineages. They also represent two different types of chemistry, racemization and transamination.

All of the catalytic bases for the three enzymes (K39 and Y265 in AlaR, K145 in DaAT, and K258 in AspAT) have highly perturbed titration curves, capable of amphoteric behavior over a wide pH range. This means that they can serve as proton acceptors and then release the proton to regenerate the base. The titration curves for these catalytic bases have similar shapes.

A number of other interesting similarities are found for the three proteins. Each of the catalytic lysine residues (K39 in AlaR, K145 in DaAT, and K258 in AspAT) has a nearby residue with a heavily downshifted pKₐ (D313′ in AlaR, E32 in DaAT and Y70′ in AspAT). All three enzymes have histidine residues with downshifted pKₐ's located near the pyridine ring of the PLP (H166 in AlaR; H100 in DaAT; and H143 and H189 in AspAT). These similarities appear to be markers for PLP-dependency.

From the point of view of function prediction, the differences between alanine racemase and the two transaminases are especially of interest. It has been noted previously that the transaminases have an acid group near the nitrogen atom of the PLP (E177 in DaAT and D222 in AspAT) whereas the residue closest to the ring nitrogen atom in alanine racemase is R219. We now find that E177 in DaAT and D222 in AspAT are characterized by strongly downshifted pKₐ's. This means that in the two transaminases, the nitrogen atom of the PLP ring is likely to remain protonated. The interaction between the pyridinium ion and the side chain carboxylate group thus may be characterized as an ion pairing interaction with the proton strongly localized on the PLP nitrogen atom. The proximity of a very-low-pKₐ acid to the pyridinium nitrogen atom therefore is likely to keep the proton inside a relatively deep potential well and the pyridinium nitrogen end of the PLP ring is therefore able to serve as an electron sink during transamination. It has been noted previously that, since the closest group to the nitrogen atom of the PLP ring in alanine racemase is R219, the ring nitrogen atom is more difficult to protonate. We now have the additional information that R219 of alanine racemase has a very upshifted pKₐ and, therefore, retains its positive charge over a wider pH range and binds the proton more strongly than a more typical arginine side group. These properties related to pH-dependent behavior appear to serve as markers for the specific types of chemistry that a B6-dependent enzyme can catalyze.

Another very interesting difference between the racemase and the two transaminases pertains to the environment of the phenolic oxygen atom on the PLP ring. In alanine racemase, the closest group to this oxygen atom in the external aldimine is the side chain of Arg136, which was found to have a very high pKₐ. Since R136 is thus expected to remain protonated over a wide pH range, the deprotonated form of the phenolic 0 atom of PLP is stabilized at physiological pH. A negatively charged phenolate on the PLP ring would tend to prevent the PLP from serving as an electron sink. Thus the environment around the phenol group of the PLP affects the PLP in the same direction as does the environment around pyridine nitrogen atom: both tend to destabilize negative charge in the PLP ring. On the other hand in the two transaminases, the closest groups to the phenolic oxygen atom of PLP are residues with very elongated titration curves (Y31 and K145 in DaAT; Y225 in AspAT). The low pKₐ's of Y31 in DaAT and Y225 in AspAT suggest that the phenolic oxygen atom of the external aldimine is protonated, which would tend to help the PLP ring serve as an electron sink. Because the phenolic oxygen atom is surrounded by soft residues, it is possible that it undergoes proton transfer during the transamination reaction. These are additional markers of the specific types of chemistry for a B6 enzyme and thus can help to identify similar chemistry in a protein of unknown function.

In this fashion, the predicted pH-dependent properties (titration curve shapes and the calculated pKₐ's) of the ionizable residues in a protein can be used to identify specific types of chemistry. We have identified markers of PLP dependence. We have also identified markers to distinguish between a racemase and a transaminase.

### REFERENCES

Antosiewicz et al. (1996) J. Comp. Chem. 17, 1633-1644.

Babbitt et al. (1998) in Encyclopedia of Computational Chemistry, ed. Schleyer, P. v. R. (Wiley, Chichester, West Sussex, U.K.), 2859-2870.

Baker et al. (2001) “Electrostatics of Nanosystems: Application to Microtubules and the Ribosome.” Proc. Natl. Acad. Sci. USA, 98, 10037-10041.

Bashford et al. (1991) J. Phys. Chem. 95, 9556-9561.

Bashford et al. (1992) J. Mol. Biol. 224, 473-486.

Berman et al. (2000) The Protein Data Bank. Nucleic Acids Res., 28(1), 235-242.

Beroza et al. (1995) “Electrostatic Calculations Of Amino Acid Titration And Electron Transfer, Q-AQB—>QAQ-B, In The Reaction Center.” Biophys. J. 68(6), 2233-2250.

Birney et al. (2001) Nature 409, 827-828.

Bode et al. (1987) “The High-Resolution X-Ray Crystal Structure Of The Complex Formed Between Subtilisin Carlsberg And Eglin C, An Elastase Inhibitor From The Leech Hirudo Medicinalis. Structural Analysis, Subtilisin Structure And Interface Geometry.” Eur. J. Biochem. 166, 673.

Bohren et al. (1994) Biochem. 33, 2021-2032.

Carlson et al.(1999) J. Med. Chem. 42, 109-117.

Carrell et al. (1994) Acta Crystallogr. D50, 5469-5480.

Carter et al. (2001) “Four-Body Potentials Reveal Protein-Specific Correlations To Stability Changes Caused By Hydrophobic Core Mutations.” J. Mol. Biol., 311(4), 625-638.

Chen et al. (2001) “Ligand-Protein Inverse Docking And Its Potential Use In The Computer Search Of Protein Targets Of A Small Molecule.” Proteins, 43(2), 217-226.

Chen (2001) “Computer Search of Putative Protein Targets of a Small Molecule.” Biophys. J., 80, 497A.

Cleasby et al.(1996) Nature Structural Biology 3, 470-479.

Coll et al. (1990) “Enzymatic mechanism of creatine amidinohydrolase as deduced from crystal structures.” J. Mol. Biol., 214, 597-610.

Coulson et al. (1970) Nature 227, 180-181.

Dennis et al. (2002) “Computational Mapping Identifies The Binding Sites Of Organic Solvents On Proteins.” Proc. Natl. Acad. Sci. USA, 99, 4290-4295.

Di Cera et al. (2003) “Serine Proteases” in Encyclopedia of the Human Genome, D. N. Cooper, Editor., Macmillan: London. p. in press.

Elcock (2001) “Prediction Of Functionally Important Residues Based Solely On The Computed Energetics Of Protein Structure.” J. Mol. Biol., 312, 885-896.

Fetrow et al. (1998) J. Mol. Biol. 281, 949-968.

Fetrow et al.(1999) FASEB J. 13, 1866-1874.

Fetrow et al.(2001) Protein Sci. 10, 1005-1014.

Fiser et al. (2000) “Modeling Of Loops In Protein Structures.” Protein Sci. 9: 1753-1773.

Fraser et al. (1995). “The Minimal Gene Complement Of Mycoplasma Genitalium.” Science 270, 397-403.

Gilson, M. K. (1993) Proteins 15, 266-282.

Gish et al. (1993) “Identification Of Protein Coding Regions By Database Similarity Search.” Nature Genetics, 3, 266-272.

Gracy et al. (1968) J. Biol. Chem. 243, 3161-3168.

Harrison et al. (1994) Biochem. 33, 2011-2020.

Hartman, F. C. (1970) Biochem. Biophys. Res. Commun. 39, 384-388.

Hegyi et al. (1999) J. Mol. Biol. 288, 147-164.

Heidelberg et al. (2000). “DNA Sequence Of Both Chromosomes Of The Cholera Pathogen Vibrio Cholerae.” Nature 406, 477-483.

Henrich et al. (2003) “The Crystal Structure Of The Proprotein Processing Proteinase Furin Explains Its Stringent Specificity.” Nat. Struct. Biol., 10, 520-526.

Holmquist (2000) “Alpha/Beta Hydrolase Fold Enzymes: Structures, Functions And Mechanisms.” Curr Protein and Peptide Sci., 1, 209-235.

Holyoak et al. (2003) “2.4 Å Resolution Crystal Structure of the Prototypical Hormone-Processing Protease Kex2 in Complex with an Ala-Lys-Arg Boronic Acid Inhibitor.” Biochem., 42, 6709-6718.

Jeffery et al. (1998). “Crystal Structure of Saccharomyces cerevisiae Cytosolic Aspartate Aminotransferase.” Protein Sci. 7, 1380-1387.

Jeffery et al. (2000). “The Role Of Residues Outside The Active Site: Structural Basis For Function Of C191 Mutants Of Escherichia Coli Aspartate Aminotransferase.” Protein Engineering 13, 105-112.

Komives et al. (1991) Biochem. 30, 3011-3019.

Kondo et al. (1994). “Cloning And Expression Of Cdna Of Human Delta 4-3-Oxosteroid 5 Beta-Reductase And Substrate Specificity Of The Expressed Enzyme.” Eur. J. Biochem. 219, 357-363.

Laskowski et al. (1996) “Protein Clefts In Molecular Recognition And Function.” Protein Sci., 5, 2438-2452.

Lavie et al. (1994) Biochem. 33, 5469-5480.

Li et al. (1998) “Crystal Structures Of Reaction Intermediates Of L-2-Haloacid Dehalogenase And Implications For The Reaction Mechanism.” J. Biol. Chem., 273, 15035-15044.

Lichtarge et al. (1996) “An Evolutionary Trace Method Defines Binding Surfaces Common To Protein Families.” J. Mol. Biol., 257(2), 342-58.

Liu et al. (2003) “The Full-Length Cdna Of S. Japonicum Genes.” EMBL GenBank DDBJ databases.

Lodi et al. (1991) Biochem. 30, 6948-6956.

Ma et al. (2001) “Protein Functional Epitopes: Hot Spots, Dynamics And Combinatorial Libraries.” Curr. Opin. Struct. Biol., 11, 364-369.

Madura et al. (1995) Comput. Phys. Commun. 91, 57-95.

Makino et al. (2003) “Genome Sequence Of Vibrio Parahaemolyticus: A Pathogenic Mechanism Distinct From That Of V. Cholerae.” Lancet 361, 743-749.

Malaisse-Lagae et al. (1989) Biochim. Biophys. Acta 998, 118-125.

Marcotte et al. (1999) “Detecting Protein Function And Protein-Protein Interactions From Genome Sequences.” Science, 285(5428), 751-753.

Marcotte et al. (1999) “A Combined Algorithm For Genome-Wide Prediction Of Protein Function.” Nature, 402(6757), 83-86.

Marcotte (2000) “Computational Genetics: Finding Protein Function By Nonhomology Methods.” Curr. Opin. Struct. Biol., 10(3), 359-365.

Miyahara et al. (1994) “X-Ray Crystallographic Study Of Pyridoxamine 5′-Phosphate-Type Aspartate Aminotransferases From Escherichia Coli In Three Forms.” J. Biochem. (Tokyo) 116, 1001.

Nelson et al. (2002) “Complete Genome Sequence And Comparative Analysis Of The Metabolically Versatile Pseudomonas Putida KT2440.” Environ. Microbiol. 4, 799-808.

Ondrechen et al. (2001) J. Am. Chem. Soc. 123, 2830-2834.

Oshiro et al. (1998) in Encyclopedia of Computational Chemistry, ed. Schleyer, P. v. R. (Wiley, Chichester, West Sussex, U. K.), 1606-1613.

Park et al. (2000) “Vibrational Stark Spectroscopy In Proteins: A Probe And Calibration For Electrostatic Fields.” Book of Abstracts, 219th ACS National Meeting, San Francisco, Calif., March 26-30, HYS-352.

Paulsen et al. (2003) “Role Of Mobile DNA In The Evolution Of Vancomycin-Resistant Enterococcus Faecalis.” Science 299, 2071-2074.

Qin et al. (1993) “Molecular Cloning Of Multiple Cdnas Encoding Human Enzymes Structurally Related To 3 Alpha-Hydroxysteroid Dehydrogenase.” J. Steroid Biochem. Mol. Biol. 46, 673-679.

Ramani et al. (2003) “Exploiting The Co-Evolution Of Interacting Proteins To Discover Interaction Specificity.” J. Mol. Biol., 327, 273-284.

Rhee et al. (2002) “Complete Genome Sequence Of Vibrio Vilnificus.” EMBL GenBank DDBJ databases.

Ridder et al. (1999) “Crystal Structures Of Intermediates In The Dehalogenation Of Haloalkanoates By L-2-Haloacid Dehalogenase.” J. Biol. Chem., 274, 30672-30678.

Sasaki et al. (2001) “Oryza Sativa Nipponbare (GA3) Genomic DNA, Chromosome 1, PAC Clone.” EMBL GenBank DDBJ databases.

Schwede et al. (2003) “SWISS-MODEL: An Automated Protein Homology-Modeling Server.” Nucleic Acids Res., 31, 3381-3385.

Shaw et al. (1997) “Determination Of The Structure Of Alanine Racemase From Bacillus Stearothermophilus At 1.9 Å Resolution.” Biochem., 36(6), 1329-1342

Sjolander (1998) “Phylogenetic Inference In Protein Superfamilies: Analysis Of SH2 Domains.” Proc. Int. Conf. Intell. Syst. Mol. Biol., 6, 165-74.

Skolnick et al. (2000) Trends Biotechnol. 18, 34-39.

Stammers et al. (1999) “2.0 Å X-Ray Structure Of The Ternary Complex Of 7,8-Dihydro-6-Hydroxymethylpterinpyrophosphokinase From Escherichia Coli With ATP And A Substrate Analogue.” FEBS Lett. 456, 49.

Stamper et al. (1998) “Reaction Of Alanine Racemase With 1-Amino-Ethylphosphonic Acid Forms A Stable External Aldimine.” Biochem., 37(29), 10438-10445

Stover et al. (2000) “Complete Genome Sequence Of Pseudomonas Aeruginosa PAO1, An Opportunistic Pathogen.” Nature 406, 959-964.

Teichmann et al. (2001) “Determination Of Protein Function, Evolution And Interactions By Structural Genomics.” Curr Opin. Struct. Biol., 11(3), 354-363.

Tetelin et al. (2000) “Complete Genome Sequence Of Neisseria Meningitidis Serogroup B Strain MC58.” Science 287, 1809-1815.

Thompson et al. (1994) “CLUSTAL W: Improving The Sensitivity Of Progressive Multiple Sequence Alignment Through Sequence Weighting, Position-Specific Gap Penalties And Weight Matrix Choice.” Nucleic Acids Res., 22(22), 4673-4680; http://www.ebi.ac.uk/clustalw.

Vriend (1990) J. Mol. Graph., 8, 52-56

Waley et al. (1970) Nature 227, 181.

Wallace et al. (1997) Protein Sci. 6, 2308-2323.

Warwicker et al. (1982) J. Mol. Biol. 157, 671-679.

Waugh et al. (2001) Pac. Symp. Biocomput. 1, 360-371.

Wells et al. (1994) Biochem. 33, 5777-5782.

Wermuth et al. (1987) “Primary Structure Of Aldehyde Reductase From Human Liver.” Prog. Clin. Biol. Res. 232, 297-307.

Wilson et al. (1992) Science 257, 81-84.

Xiao et al. (1999) “Crystal Structure Of 6-Hydroxymethyl-7,8-Dihydropterin Pyrophosphokinase, A Potential Target For The Development Of Novel Antimicrobial Agents.” Structure Fold Des. 7, 489-496.

Yamamoto et al. (1992) “Crystal Structure Of Papain-Succinyl-Gln-Val-Val-Ala-Ala-P-Nitroanilide Complex At 1.7-A Resolution: Noncovalent Binding Mode Of A Common Sequence Of Endogenous Thiol Protease Inhibitors.” Biochem., 31, 11305-11309.

Yang et al. (1993) PROTEINS: Structure, Function, and Genetics 15, 252-263.

Yao et al. (2003) “An Accurate, Sensitive, And Scalable Method To Identify Functional Sites In Proteins.” J. Mol. Biol., 326, 255-261.

Zhang et al. (1994) Biochemistry 33, 2830-2837.

Zhou et al. (1998) “Transition State Structure Of Arginine Kinase: Implications For Catalysis Of Bimolecular Reactions.” Proc. Natl. Acad. Sci. USA, 95, 8449-8454.

While the present invention has been described in conjunction with a preferred embodiment, one of ordinary skill, after reading the foregoing specification, will be able to effect various changes, substitutions of equivalents, and other alterations to the compositions and methods set forth herein. It is therefore intended that the protection granted by Letters Patent hereon be limited only by the definitions contained in the appended claims and equivalents thereof.

What is claimed is:

1. A method for determining interaction sites in a protein, said method comprising the steps of:
identifying in said protein ionizable amino acid residues having anomalous predicted theoretical microscopic titration curve behavior; and
clustering certain of said identified ionizable amino acid residues into putative interaction sites.

2. The method of claim 1, wherein said identifying and clustering steps comprise the steps of:
(a) obtaining a three-dimensional structure of said protein;
(b) calculating an electrical potential function for said protein structure;
(c) calculating a titration curve for each ionizable residue in said protein;
(d) evaluating the shape of said titration curve for each said residue;
(e) identifying any said residue with a perturbed titration curve in comparison with other residues of the same kind; and
(f) identifying any said perturbed titration curve residues that are in a cluster, wherein the existence of said cluster indicates that the residues in said cluster are at an interaction site in said protein.

3. The method of claim 2, further comprising the step of identifying any said perturbed titration curve residues that do not fall into any said cluster.

4. The method of claim 2, wherein, in step (a), the three-dimensional structure of said protein is known.

5. The method of claim 2, wherein, in step (a), the three-dimensional structure of said protein is theoretically determined from the amino acid sequence of said protein.

6. The method of claim 2, wherein, in step (f), said residues are identified as being in a cluster by virtue of being located in physical proximity to each other in said three-dimensional structure of said protein.

7. The method of claim 5, wherein said physical proximity has a distance of less than 15 Å.

8. The method of claim 5, wherein said physical proximity has a distance of less than 10 Å.

9. The method of claim 5, wherein said physical proximity has a distance of less than 7 Å.

10. The method of claim 5, wherein said physical proximity has a distance of approximately 6 Å or less.

11. The method of claim 2, wherein said perturbed titration curve is an elongated titration curve where partial protonation is predicted to extend over a wide pH range.

12. The method of claim 2, wherein said step of identifying said residue with a perturbed titration curve can be performed by visual inspection, statistical analysis or automated classification.

13. The method of claim 2, wherein said interaction site is further identified as a catalytic site.

14. The method of claim 2, wherein said interaction site is further identified as a recognition site.

15. The method of claim 2, wherein said interaction site is further identified as a cofactor binding site.

16. The method of claim 2, wherein said interaction site is further identified as a ligand binding site.
